SEQUENCE LISTING (17 sequences)

SEQ ID NO: 1 - protein, 21 aa, Homo sapiens
LNTGSGKTDECHYVQSKGRAR

SEQ ID NO: 2 - protein, 21 aa, Artificial Sequence
  Description of Artificial Sequence Note = Synthetic Construct
LGTGSGKTDECHYVQSKGRAR

SEQ ID NO: 3 - protein, 21 aa, Artificial Sequence
  Description of Artificial Sequence Note = Synthetic Construct
MRTGAGKTDECHYVQSKGRAR

SEQ ID NO: 4 - protein, 21 aa, Drosophila melanogaster
LGHRSSKEEDCHYVQCKGRAR

SEQ ID NO: 5 - protein, 21 aa, Drosophila melanogaster
LPTGSGKTDECHYVQSKGRAR

SEQ ID NO: 6 - DNA, 42 bp, Artificial Sequence
  Description of Artificial Sequence Note = Synthetic Construct
gggaagcucagaauauugcacaaguagagcuucucgaucccc

SEQ ID NO: 7 - DNA, 40 bp, Artificial Sequence
  Description of Artificial Sequence Note = Synthetic Construct
cccuucgagucuuauaacguguucaucucgaagagcuagg

SEQ ID NO: 8 - DNA, 42 bp, Artificial Sequence; identical to SEQ ID NO: 6
  Description of Artificial Sequence Note = Synthetic Construct

SEQ ID NO: 9 - DNA, 42 bp, Artificial Sequence
  Description of Artificial Sequence Note = Synthetic Construct
cccuucgagucuuauaacguguucaucucgaagagcuagggg

SEQ ID NO: 10 - DNA, 40 bp, Artificial Sequence
  Description of Artificial Sequence Note = Synthetic Construct
gggaagcucagaauauugcacaaguagagcuucucgaucc

SEQ ID NO: 11 - DNA, 42 bp, Artificial Sequence; identical to SEQ ID NO: 9
  Description of Artificial Sequence Note = Synthetic Construct

SEQ ID NO: 12 - DNA, 27 bp, Artificial Sequence
  Description of Artificial Sequence Note = Synthetic Construct
ggcaaugaaagacggugagcuggugau

SEQ ID NO: 13 - DNA, 27 bp, Artificial Sequence
  Description of Artificial Sequence Note = Synthetic Construct
gguauucacuccagagcgaugaaaacg

SEQ ID NO: 14 - DNA, 42 bp, Artificial Sequence; identical to SEQ ID NO: 6
  Description of Artificial Sequence Note = Synthetic Construct

SEQ ID NO: 15 - DNA, 42 bp, Artificial Sequence
  Description of Artificial Sequence Note = Synthetic Construct
ggggaucgagaagcucuacuugugcaauauucugagcuuccc

SEQ ID NO: 16 - DNA, 40 bp, Artificial Sequence; identical to SEQ ID NO: 10
  Description of Artificial Sequence Note = Synthetic Construct

SEQ ID NO: 17 - DNA, 40 bp, Artificial Sequence
  Description of Artificial Sequence Note = Synthetic Construct
ggaucgagaagcucuacuugugcaauauucugagcuuccc

(19) United States
(12) Patent Application Publication
(10) Pub. No.: US 2015/0182601 A1
(43) Pub. Date: Jul. 2, 2015

(54) COMPOSITIONS AND METHODS FOR SELECTIVELY PRODUCING SIRNA

(71) Applicant: University of Utah Research Foundation, Salt Lake City, UT (US)

(72) Inventors: Noah C. Welker, Salt Lake City, UT (US); Tuhin S. Maity, Los Alamos, NM (US); Phillip Joseph Aruscavage, JR., Salt Lake City, UT (US); Brenda L. Bass, Salt Lake City, UT (US)

(73) Assignee: University of Utah Research Foundation, Salt Lake City, UT (US)

(21) Appl. No.: 14/444,828

(22) Filed: Jul. 28, 2014

Related U.S. Application Data
(63) Continuation of application No. 13/642,417, filed on Feb. 28, 2013, now abandoned.
(60) Provisional application No. 61/327,046, filed on Apr. 22, 2010.

(51) Int. Cl. A61K 38/46; A61K 31/713

(52) U.S. Cl. CPC A61K 38/465; A61K 31/713; C12Y 301/26003

(57) ABSTRACT

The disclosure provided herein provides compositions and methods for producing siRNA. Also disclosed are compositions and methods for modulating the production of siRNA. Also disclosed herein are compositions and methods of treating a disease in a subject comprising administering a Dicer enzyme comprising a helicase domain or a mutated Dicer enzyme to a subject. Also disclosed herein are methods of screening for a candidate modulator that modulates siRNA production.

### CROSS-REFERENCE TO RELATED PATENT APPLICATIONS

This application is a continuation application of U.S. patent application Ser. No. 13/642,417 filed Feb. 28, 2013, which is a national phase application of PCT/US2011/033600 filed Apr. 22, 2011, which claims the benefit of priority to U.S. Provisional Application No. 61/327,046 filed Apr. 22, 2010, each application of which is hereby incorporated herein by reference in its entirety.

### STATEMENT REGARDING FEDERALLY FUNDED RESEARCH

This invention was made with government support under Grant No. GM067106 awarded by the National Institute of General Medical Sciences. The government has certain rights in the invention.

### FIELD OF THE INVENTION

The present invention relates generally to the fields of molecular biology. More specifically, the invention concerns methods and compositions useful for producing siRNA. The invention also concerns methods and compositions useful for modulating the production of siRNA as well as treating a disease in a subject comprising administering a Dicer enzyme, helicase domain, or a mutated Dicer enzyme, to a subject. The invention also concerns methods and compositions useful for screening for a candidate modulator of siRNA production.

### REFERENCE TO SEQUENCE LISTING

The Sequence Listing submitted Nov. 5, 2014 as a text file named “21101_{—}0243U3_Sequence_Listing.txt,” which was created on Nov. 5, 2014 and has a size of 4,798 bytes is hereby incorporated by reference pursuant to 37 C.F.R. §1.52(e)(5).

### SUMMARY

Disclosed herein are methods of producing siRNA in vitro comprising: introducing at least one dsRNA molecule and at least one Dicer enzyme comprising a helicase domain to a sample to form a reaction mixture, wherein the dsRNA comprises blunt ends or at least one 5′ overhang; and incubating the reaction mixture for a time sufficient to produce siRNA.

Also, disclosed herein are methods of producing siRNA in vitro comprising: introducing at least one dsRNA molecule and at least one Dicer enzyme comprising a helicase domain to a sample to form reaction mixture, wherein the dsRNA comprises blunt ends or at least one 5′ overhang; and incubating the reaction mixture for a time sufficient to produce siRNA, further comprising introducing ATP to the reaction mixture.

Also, disclosed herein are methods of producing siRNA in vitro comprising: introducing at least one dsRNA molecule and at least one Dicer enzyme comprising a helicase domain to a sample to form a reaction mixture, wherein the dsRNA comprises blunt ends or at least one 5′ overhang; and incubating the reaction mixture for a time sufficient to produce siRNA.

Also, disclosed herein are methods of producing siRNA in vitro comprising: introducing at least one dsRNA molecule and at least one Dicer enzyme comprising a helicase domain to a sample to form a reaction mixture, wherein the dsRNA comprises blunt ends or at least one 5′ overhang; and incubating the reaction mixture for a time sufficient to produce siRNA, wherein the method selectively produces siRNA.

Also disclosed herein are methods of modulating the production of siRNA in a subject comprising: administering a mutated Dicer enzyme to the subject, wherein the Dicer enzyme comprises a mutation in the helicase domain of the Dicer enzyme, thereby modulating the production of siRNA in the subject.

Also disclosed herein are methods of modulating the production of siRNA in a subject comprising: administering a Dicer enzyme comprising a helicase domain to the subject, thereby modulating the production of siRNA in the subject.

Also disclosed herein are methods of treating a disease in a subject comprising: administering a mutated Dicer enzyme to the subject, wherein the Dicer enzyme comprises a mutation in the helicase domain, thereby treating the subject.

Also disclosed herein are methods of treating a disease in a subject comprising: administering a Dicer enzyme comprising a helicase domain to the subject, thereby treating the subject.

Also disclosed herein are methods of screening for a candidate modulator that modulates siRNA production comprising the steps of: (a) determining the production of siRNA in a sample comprising at least one dsRNA molecule and at least one Dicer enzyme comprising a helicase domain; (b) exposing the sample comprising at least one dsRNA molecule and at least one Dicer enzyme comprising a helicase domain to the candidate modulator; (c) determining the effect of the candidate modulator on the production of siRNA in the sample; wherein a change in the production of siRNA in the sample after exposure to the candidate modulator is indicative of a modulator of siRNA production.

Also disclosed herein are methods of screening for a candidate modulator that modulates microRNA production comprising the steps of: (a) determining the production of microRNA in a sample comprising at least one dsRNA molecule and at least one Dicer enzyme comprising a helicase domain, wherein the Dicer enzyme comprises a mutation in the helicase domain; (b) exposing the sample comprising at least one dsRNA molecule and at least one Dicer enzyme comprising a mutation in the helicase domain to the candidate modulator; (c) determining the effect of the candidate modulator on the production of microRNA in the sample; wherein a change in the production of microRNA in the sample after exposure to the candidate modulator is indicative of a modulator of microRNA production.

### BRIEF DESCRIPTION OF THE DRAWINGS

The accompanying drawings, which are incorporated in and constitute a part of this specification, illustrate several embodiments of the invention and together with the description, serve to explain the principles of the invention. These are non-limiting examples.

The file of this patent contains at least one photograph or drawing executed in color. Copies of this patent with color drawing(s) or photograph(s) will be provided by the Patent and Trademark Office upon request and payment of necessary fee.

For a fuller understanding of the nature and advantages of the present invention, reference should be had to the following detailed description taken in connection with the accompanying drawings, in which:

FIGS. 1A and 1B depict that Dicer's helicase domain has well-conserved motifs. (A) Domain organization of C. elegans DCR-1 and Drosophila Dicer-1 and Dicer-2, color coded to indicate: helicase domain, domain of unknown function 283 (DUF283), Piwi Argonaute Zwille domain (PAZ), RNase III domains, and the dsRNA binding motif (dsRBM). (B) Amino acids within the highly conserved Motif I (Walker A), Motif II (Walker B), and Motif VI are shown, with mutants analyzed in this study indicated. Underlined amino acids show residues mutated in previous studies and discussed in text.

FIGS. 2A, 2B, 2C, 2D, and 2E depict_that the helicase domain of C. elegans Dicer is required for cleavage of dsRNA with blunt or 5′ overhanging termini, but not 3′ overhanging termini. (A) PhosphorImage showing production of mature let-7 miRNA (let-7), from 5′ ³²P-end-labeled (*) pre-let-7, incubated for indicated times without (-), or with, WT or ΔH C. elegans extract in extract cleavage buffer (12 mM MgOAc; Experimental Procedures). M, positions of RNA decade ladder and 22 nt miRNA. (B) Data from multiple assays as in (A) were quantified to determine % miRNA (100×[radioactivity in miRNA band/total radioactivity in lane]); miRNA band included radioactivity in major and two immediately adjacent bands, but plots did not differ significantly when only predominant band was monitored. Data points show average values; error bars, standard error of the mean; n=2. (C) PhosphorImage of reaction products generated after 60 min incubation of 40/42 dsRNAs with indicated termini, without (-), or with, WT or AH extract in extract cleavage buffer (10 mM MgOAc; Experimental Procedures). As cartooned, 40/42 dsRNAs were 5′ ³²P-end-labeled on bottom strand. Products generated by Dicer measuring from constant or variable termini are indicated. Marked lengths (left) were determined with reference to alkaline hydrolysis (AH) ladders, 10 nt RNA ladders (10 nt), and T1 ribonuclease ladders (FIG. 7). (D) Data from multiple assays as in (C) were quantified to compare cleavage from three types of termini. % siRNA=100×(radioactivity in siRNA band/total radioactivity in lane); radioactivity in siRNA band included predominant and immediately adjacent bands if present. % siRNA values for BLT end processing included constant ends of 3′-ovr and 5′-ovr dsRNAs as well as both termini of BLT-BLT dsRNAs. Error bars, standard error of the mean (n≦6). (E) Schematic of observed cleavage sites in 40/42 dsRNAs reacted as in (C) with extracts of wildtype C. elegans. Sites were determined using data for 40/42 dsRNAs labeled on the top (FIG. 7) or bottom (FIG. 2C) strand. Length in nts is shown above arrows marking major (long arrows) and minor (short arrows) cleavage with respect to Dicer measurement from constant blunt end (red) or variable end (blue).

FIGS. 3A, 3B, and 3C show dsRNA with blunt termini give rise to more internally derived siRNAs than those with 3′ overhangs. (A) PhosphorImages of northern blots comparing the reaction of 106 BLT-BLT and 3′ovr-3′ovr dsRNA incubated with WT and AH extracts for various times, in extract cleavage buffer (10 mM MgOAc; Experimental Procedures). Cartoon shows relative position of probes designed to detect siRNAs generated from termini (top and bottom panels) or middle (middle panel) of dsRNAs. Asterisks denote intermediates; each asterisk represents one cleavage event (*˜80 nt, **˜57 nt, ***˜34 nt). Marked lengths (nts) were determined from data of FIG. 7. (B) Data from multiple analyses as in (A) were quantified to determine the average % siRNA (see FIG. 2D) for a 60 min incubation with dsRNAs and extracts indicated. Conditions were as in (A) except some reactions contained 6.5 mM MgOAc (control experiments showed no difference in reactions with 6.5 or 10 mM MgOAc). When siRNA bands were heterogeneous, siRNA radioactivity included all proximal bands. Error bars, standard error of the mean (n>3). (C) Northern blots as in (A) show reaction of 106 BLT-BLT dsRNA incubated for 60 min with WT extract (Ext.) in extract cleavage buffer modified to contain varying amounts of ATP (mM), MgOAc (mM) and EDTA. ATP stimulates cleavage and is required for accumulation for internally derived siRNAs (overexposure of middle panel, FIG. 8). Asterisks, cleavage intermediates as in (A). Marked lengths (nts) were determined from data of FIG. 7.

FIGS. 4A, 4B, 4C, and 4D show that purified Drosophila Dicer-2 discriminates duplex termini. (A) Coomassie-stained SDS-PAGE of WT Drosophila Dicer-2 and point mutants after purification to homogeneity. (B) PhosphorImage of products separated by 17% denaturing PAGE after a 30 min incubation of 0.8 nM ³²P-end-labeled (bottom strand) 40/42 dsRNAs (as in FIG. 2C) in cleavage buffer without Dicer (-) or containing 30 nM wildtype Dicer-2 or helicase mutants as indicated. All reactions contained 8 mM ATP except those lacking all ATP (-ATP). (C) PhosphorImage of northern blot showing products generated from 1 nM 106 BLT-BLT or 3′ovr-3′ovr dsRNAs incubated in cleavage buffer (60 min) without Dicer (-), or with 10 nM Drosophila Dicer-2, WT or mutant as indicated. All reactions contained 5 mM ATP; probes were as in FIG. 3A. (D) Data from multiple northern blot analyses as in (C), with WT Drosophila Dicer-2 and incubation times as indicated, were quantified to determine ratio of siRNAs derived from internal cleavage events to those derived from 5′ and 3′ termini. % siRNA, as in FIG. 2D; error bars, standard error of the mean (n>3).

FIGS. 5A, 5B, 5C, and 5D show that Dicer-2 exhibits two modes of cleavage, depending on dsRNA termini. (A) PhosphorImage of products resolved by 12% denaturing PAGE after incubation of 0.5 nM ³²P-internally-labeled 104 bp dsRNA in cleavage buffer (30 min; 24° C.) with varying WT Dicer-2 and 5 mM ATP. M, RNA decade markers, labeled for length (nts) by comparison to AH and P1 nuclease products of end labeled ³²P-104 bp dsRNA. Mobility of full-length dsRNA (FL) and siRNA are marked. (B) As in (A) except WT Dicer-2 was constant (80 nM), reactions were on ice, and reaction time was varied. Asterisks, intermediates with one (*), two (**) or three (***) cleavages. 0*, aliquots taken immediately (≦10 seconds) after starting reaction with ATP. (C-D) Quantification of multiple “trap” experiments using 80 nM Dicer-2 and 0.5 nM ³²P-internally labeled 106 BLT-BLT (C) or 106 3′ovr-3′ovr (D) dsRNA. All reactions were initiated by adding ATP (5 mM) followed shortly (30″,106 BLT-BLT; 5′,106 3′ovr-3′ovr) by the addition of cold trap (2000 nM 82 BLT-BLT; squares), no trap (circles), or ³²P-internally labeled dsRNA if 82 BLT-BLT trap was added first (open diamonds). Data points show (number of counts in siRNA/total counts in reaction)*100; error bars, standard deviation (n≧2); non-visible error bars, standard deviation <0.25%. Data were fit to a pseudo first order equation: Fraction product=a*(1-exp^{-kt})+b; a=amplitude of rate curve, b=baseline (˜0), k=pseudo first order rate constant, t=time. Shading boundary marks trap addition.

FIG. 6. Model for distributive and processive cleavage of dsRNA by Dicer. Two steps, end recognition and cleavage, are shown for dsRNA with 3′ overhanging termini (top left), or blunt termini (top right) reacting with Dicer (shown with color-coded domains). A common intermediate is shown for end recognition (dots, nts of blunt dsRNA); this step is ATP-independent for dsRNA with 3′ overhanging termini, but for blunt termini requires ATP to facilitate access to the 3′ end by the PAZ domain. Subsequently, cleavage of either dsRNA produces siRNA (red font, lengths based on C. elegans DCR-1). In the Distributive mode Dicer dissociates following cleavage and subsequent cleavage events require rebinding. In the Processive mode Dicer does not dissociate, but undergoes a conformational change that engages the helicase domain for translocation, and processive cleavage. siRNAs with 3 nt overhangs, but 4 nt overhangs were also observed for C. elegans extracts.

FIGS. 7A, 7B, 7C and 7D. (A) PhosphorImage of products resolved by 17% denaturing PAGE, following 60′ incubation of BLT-3′ ovr (B-3′ovr), BLT-BLT (B-B), or BLT-5′ovr (B-5′ovr) 40/42 dsRNAs without (-) or with (WT) extract as in FIG. 2C. dsRNAs were 5′ ³²P-end-labeled on top strand (black) as pictured in FIG. 2C. Arrows indicate non-specific RNA degradation present in the—extract control lane, particularly evident in BLT-5′ovr. AH, alkaline hydrolysis; 10 nt, 10 nt RNA ladders (Ambion). Lengths in nts are marked on left. (B) PhosphorImage of products resolved by 17% denaturing PAGE, resulting from cleavage as in (A), of BLT-5′ovr 40/41 dsRNAs with 1 nt 5′ overhangs that vary by terminal overhanging nucleotide (B-5′ovr_A, B-5′ovr_U, B-5′ovr_C, B-5′ovr_G). dsRNAs were 5′ ³²P-end-labeled on the bottom strand. AH, alkaline hydrolysis, length in nt marked on left. (C, D) PhosphorImage showing RNAs resolved by 17% denaturing PAGE after various treatments of sense (S) and antisense (A) 5′ ³²P-end-labeled RNAs used to form 106 (C) and 40/42 (D) dsRNAs. T1, T1 ribonuclease; AH, alkaline hydrolysis; 10 nt, RNA ladder (Ambion). Below each PhosphorImage are sequences of RNAs used to make various dsRNAs indicated in parentheses. All RNAs were sequenced, and in a subset guanosines are color-correlated with red and green boxes to illustrate bands derived from limited cleavage with T1 ribonuclease. Due to sequence specific effects, in all cases, our RNAs migrated slightly slower than the same length of commercially obtained RNA ladder. T1 ribonuclease ladder was prepared by incubating 10 fmol of 5′ ³²P-end labeled single-stranded RNA and 10 μg of non-specific RNA with 0.01 U RNase T1 (Ambion) in 10 μl 20 mM NaOAc (pH 4.6) at 60° C. for 15 min, followed by organic extraction and ethanol precipitation.

FIG. 8. Overexposure of northern blot shown in FIG. 3C, middle panel, to confirm the lack of internal cleavage products in the absence of ATP. Ext., WT extract; ATP and Mg⁺² concentrations are given in mM.

FIGS. 9A, 9B, and 9C show the affinity of mutant proteins to dsRNA.

### DETAILED DESCRIPTION OF THE INVENTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

The present disclosure is directed to the activity of Dicer's helicase domain which is required in vivo for processing certain endogenous short interfering RNA (siRNA), but not micro RNA (miRNA). Using C. elegans extracts, or purified Drosophila Dicer-2, activities of wild-type enzymes were compared with those with mutations in the helicase domain. The helicase domain is essential for recognition of double stranded RNA (dsRNA) with blunt or 5′ overhanging termini, but not dsRNA with 3′ overhangs, as found on miRNA precursors. For processing of long dsRNA, blunt termini, but not 3′ overhangs, lead to increased siRNAs from internal regions of the dsRNA; this activity is dependent on ATP and a functional helicase domain. dsRNA with blunt or 5′ overhanging termini engage the helicase domain for translocation and processive cleavage, an activity suited for processing long siRNA precursors of low abundance, but not necessary for the single cleavage required for miRNA processing.

One aspect of the present disclosure provides that the helicase domain of Dicer is required for distinguishing between different dsRNA termini. In another embodiment, the enzyme is engaged for processive cleavage of siRNA from internal regions of dsRNA. In yet another embodiment, the helicase domain is essential for in vivo accumulation of certain endo-siRNA, but not miRNA.

One aspect of the present disclosure therefore provides isolated siRNA comprising short double-stranded RNA from about 17 nucleotides to about 30 nucleotides in length, and in certain embodiments from about 19 to about 25 nucleotides in length, that are targeted to the target mRNA. The siRNA comprise a sense RNA strand and a complementary antisense RNA strand annealed together by standard Watson-Crick base-pairing interactions (hereinafter “base-paired”). As is described in more detail below, one strand comprises a nucleic acid sequence that is complementary or partially complementary to a target sequence contained within the target mRNA.

Embodiments of the current disclosure provide for an isolated molecule comprising dsRNA having at least one blunt ended terminus and capable of engaging the helicase domain of an enzyme for translocation and processive cleavage. In one aspect, the dsRNA is effective in treating disease in mammals. In some aspects, the dsRNA with blunt ends can serve as a substrate for siRNA production. On some aspects the dsRNA with blunt ends can serve as a substrate for siRNA production for more efficient production of siRNA.

Another embodiment of the current disclosure is directed to an isolated molecule comprising dsRNA having at least one 5′ overhanging terminus and capable of engaging the helicase domain of an enzyme for translocation and processing cleavage. In some aspects, the dsRNA having at least one 5′ overhanging terminus and capable of engaging the helicase domain of an enzyme for translocation and processing cleavage can serve as a substrate for siRNA production. On some aspects the dsRNA having at least one 5′ overhanging terminus and capable of engaging the helicase domain of an enzyme for translocation and processing cleavage can serve as a substrate for siRNA production for more efficient production of siRNA.

Also disclosed herein are methods of producing siRNA in vitro comprising: introducing at least one dsRNA molecule and at least one Dicer enzyme comprising a helicase domain to a sample to form a reaction mixture, wherein the dsRNA comprises blunt ends or at least one 5′ overhang; and incubating the reaction mixture for a time sufficient to produce siRNA.

Also disclosed herein are methods of modulating the production of siRNA in a subject comprising: administering a mutated Dicer enzyme to the subject, wherein the Dicer enzyme comprises a mutation in the helicase domain of the Dicer enzyme, thereby modulating the production of siRNA in the subject.

Also disclosed herein are methods of modulating the production of siRNA in a subject comprising: administering a Dicer enzyme comprising a helicase domain to the subject, thereby modulating the production of siRNA in the subject.

Also disclosed herein are methods of treating a disease in a subject comprising: administering a mutated Dicer enzyme to the subject, wherein the Dicer enzyme comprises a mutation in the helicase domain of the Dicer enzyme, thereby treating the subject.

Also disclosed herein are methods of treating a disease in a subject comprising: administering a Dicer enzyme comprising a helicase domain to the subject, thereby treating the subject.

Also disclosed herein are methods of screening for a candidate modulator that modulates siRNA production comprising the steps of: (a) determining the production of siRNA in a sample comprising at least one dsRNA molecule and at least one Dicer enzyme comprising a helicase domain; (b) exposing the sample comprising at least one dsRNA molecule and at least one Dicer enzyme comprising a helicase domain to the candidate modulator; (c)

determining the effect of the candidate modulator on the production of siRNA in the sample; wherein a change in the production of siRNA in the sample after exposure to the candidate modulator is indicative of a modulator of siRNA production.

Also disclosed herein are methods of screening for a candidate modulator that modulates microRNA production comprising the steps of: (a) determining the production of microRNA in a sample comprising at least one dsRNA molecule and at least one Dicer enzyme comprising a helicase domain, wherein the Dicer enzyme comprises a mutation in the helicase domain; (b) exposing the sample comprising at least one dsRNA molecule and at least one Dicer enzyme comprising a mutation in the helicase domain to the candidate modulator; (c) determining the effect of the candidate modulator on the production of microRNA in the sample; wherein a change in the production of microRNA in the sample after exposure to the candidate modulator is indicative of a modulator of microRNA production.

Also disclosed herein are methods of modulating the production of siRNA in a subject comprising: administering a Dicer enzyme comprising a helicase domain to the subject, thereby modulating the production of siRNA in the subject.

Also disclosed herein are methods of modulating the production of siRNA in a subject comprising: administering a Dicer enzyme comprising a helicase domain to the subject, thereby modulating the production of siRNA in the subject without affecting miRNA production.

Also disclosed herein are methods of treating a disease in a subject comprising: administering a mutated Dicer enzyme to the subject, wherein the Dicer enzyme comprises a mutation in the helicase domain of the Dicer enzyme, thereby treating the subject.

Also disclosed herein are methods of treating a disease in a subject comprising: administering a Dicer enzyme comprising a helicase domain to the subject, thereby treating the subject.

### DEFINITIONS AND NOMENCLATURE

It must also be noted that as used herein and in the appended claims, the singular forms “a”, “an”, and “the” include plural reference unless the context clearly dictates otherwise. Thus, for example, reference to a “dsRNA molecule” is a reference to one or more dsRNA molecules and equivalents thereof known to those skilled in the art, and so forth. For example, reference to a “dsRNA molecule” can mean one or more dsRNA molecules with a 3′ overhang, a 5′ overhang, or blunt ends and of the same or different sequence or any combination thereof.

Reference to a “Dicer enzyme” is a reference to one or more Dicer enzymes and equivalents thereof known to those skilled in the art, and so forth. For example, reference to a “Dicer enzyme” can mean one or more Dicer enzymes comprising a helicase domain or one or more Dicer enzymes that do not contain a helicase domain or one or more Dicer enzymes comprising a helicase domain wherein there is a mutation in the helicase domain or any combination thereof.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. For example, the term “about” can mean plus or minus 10% of the numerical value of the number with which it is being used. Therefore, about 50% means in the range of 45%-55%.

In this specification and in the claims which follow, reference will be made to a number of terms which shall be defined to have the following meanings: “Optional” or “optionally” means that the subsequently described event or circumstance may or may not occur, and that the description includes instances where the event or circumstance occurs and instances where it does not.

The word “or” as used herein means any one member of a particular list and also includes any combination of members of that list.

As used throughout, by “subject” is meant an individual. For example, a “subject” can be a mammal such as a primate, and, more preferably, a human. The term “subject” includes domesticated animals, such as cats, dogs, etc., livestock (e.g., cattle, horses, pigs, sheep, goats, etc.), and laboratory animals (e.g., mouse, rabbit, rat, guinea pig, etc.). For example, the subject is an animal. In certain embodiments, the subject is a human being.

As used herein, a “subject” is the same as a “patient,” and the terms can be used interchangeably.

As used herein, a “sample” or “biological sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, a “mutagen” can be a physical or chemical agent that changes the genetic material, usually DNA, of a subject and thus increases the frequency of mutations above the natural background level. In one aspect, a mutagen can be a chemical compound. In one aspect a mutagen can be a physical mutagen such as ionizing radiation. Mutagens can be divided into different categories according to their effect on DNA replication. A mutagen can act as base analogs and get inserted into the DNA strand during replication in place of the natural substrates, it can react with DNA and cause structural changes that lead to miscopying of the template strand when the DNA is replicated, or it can work indirectly by causing the cells to synthesize chemicals that have the direct mutagenic effect.

For example, and not to be limiting, a mutagen can be N-ethyl-N-nitrosourea (ENU), nitrous acid, hydroxylamine, an alkylating agent such as EMS/MMS(ethyl/methyl methyl sulphonate), an aromatic arylating agent such as DMBA, an intercalating agent such as an acridine dye (acridine orange/yellow) or ethidium bromide, a base analog such as 5-bromouracil or 2-aminopurine, TEM (tri ethylene melamine), sodium azide, non-ionizing radiations such as UV rays, or ionizing radiations such as X-rays, cosmic rays, α-particles, β-particles, or gamma rays, or DNA insertional events such as DNA transposition or DNA viral sequence insertion into genomic DNA.

As used herein, a “mutation” can be a genetic change in a gene or a subject. For example, “a mutation in the helicase domain” can mean a genetic alteration in the gene sequence that encodes the helicase domain of a Dicer enzyme. As used herein, “a mutation in the helicase domain” results in partial or complete modulation of the helicase domain of a Dicer enzyme's ability to hybridize to a dsRNA comprising blunt ends or a 5′ overhang.

By “modulate” is meant to alter, by increase or decrease.

As used herein, a “modulator” can mean a composition that can either increase or decrease the expression or activity of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition. For example, a “candidate modulator” can be an active agent or a therapeutic agent.

The term “active agent” or “therapeutic agent” is defined as an active agent, such as drug, chemotherapeutic agent, chemical compound, etc. For example, and not to be limiting an active agent or a therapeutic agent can be a naturally occurring molecule or may be a synthetic compound, including, for example and not to be limiting, a small molecule (e.g., a molecule having a molecular weight <1000), a peptide, a protein, an antibody, or a nucleic acid, such as an siRNA or an antisense molecule. An active or therapeutic agent can be used individually or in combination with any other active or therapeutic agent.

By “prevent” is meant to minimize the appearance or development of or to inhibit the occurrence of an event. For example, “prevent” can mean to minimize the appearance or development of a disease caused or exacerbated by endogenous siRNA production in a subject. For example, prevention can be 10%, 20%, 30%, 40%, 50%, 60%, 70%, 80%, 90%, 95%, 99%, 100% or any percentage in between as compared to a control

As used herein, the term “gene” refers to polynucleotide sequences which encode protein products and encompass RNA, mRNA, cDNA, single stranded DNA, double stranded DNA and fragments thereof. Genes can include introns and exons and non-coding sequences that indirectly modulate the function of other sequences. It is understood that the polynucleotide sequences of a gene can include complimentary sequences (e.g., cDNA).

The term “gene sequence(s)” refers to gene(s), full-length genes or any portion thereof “Gene sequences” can include natural genes or synthetic genes, or genes created through manipulation.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

“Peptide” as used herein refers to any peptide, oligopeptide, polypeptide, gene product, expression product, or protein. A peptide is comprised of consecutive amino acids. The term “peptide” encompasses naturally occurring or synthetic molecules.

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the peptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

“Differential expression” or different expression” as used herein refers to the change in expression levels of genes, and/or proteins encoded by said genes, in cells, tissues, organs or systems upon exposure to an agent. As used herein, differential gene expression includes differential transcription and translation, as well as message stabilization. Differential gene expression encompasses both up- and down-regulation of gene expression.

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

By “specifically hybridizes” is meant that a probe, primer, or oligonucleotide recognizes and physically interacts (that is, base-pairs) with a substantially complementary nucleic acid (for example, a an siRNA molecule) under high stringency conditions, and does not substantially base pair with other nucleic acids.

Throughout the description and claims of this specification, the word “comprise” and variations of the word, such as “comprising” and “comprises,” means “including but not limited to,” and is not intended to exclude, for example, other additives, components, integers or steps.

As used herein, an “effective amount” of the dsRNA, siRNA or miRNA is an amount sufficient to cause RNAi-mediated degradation of the target mRNA in cell.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

As used herein, “target mRNA” means an mRNA comprising a complementary sense sequence to an siRNA antisense strand. Such a target mRNA need not be 100% homologous to the siRNA antisense strand, as long as the siRNA functions to silence or otherwise form a RISC complex with the target mRNA.

As used herein the term “partially non-complementary” is intended to mean an siRNA sequence which although, perhaps sharing some sequence homology to a non-target sequence still differs sufficiently such that RNA silencing does not occur for the non-target sequence. Partially non-complementary include sequences that are 90% homologous, 85%, homologous, 80% homologous, 75% homologous, 70% homologous, 65% homologous, 60%, homologous, 55% homologous, 50% homologous, 45% homologous, 40% homologous, 35%, homologous, 30% homologous, 25% homologous, 20% homologous, 15% homologous, 10%, homologous, 5% homologous, 2% homologous, and 1% homologous to a non-target sequence. A sequence that is entirely non-homologous to a non-target sequence is considered non-complementary to the sequence.

As used herein, a “3′ overhang” refers to at least one unpaired nucleotide extending from the 3′-end of a duplexed RNA strand.

As used herein, a “5′ overhang” refers to at least one unpaired nucleotide extending from the 5′-end of a duplexed RNA strand.

As used herein, a “blunt end” or “blunt ended” refers to a duplexed RNA strand that does not comprise an unpaired nucleotide extending from the 5′-end or 3′-end of the duplexed RNA strand. A dsRNA molecule that comprises “blunt-ends” is a dsRNA molecule where both strands of the dsRNA terminate in a base pair.

Unless otherwise indicated, all nucleic acid sequences herein are given in the 5′ to 3′ direction. Also, all deoxyribonucleotides in a nucleic acid sequence are represented by capital letters (e.g., deoxythymidine is “T”), and ribonucleotides in a nucleic acid sequence are represented by lower case letters (e.g., uridine is “u”).

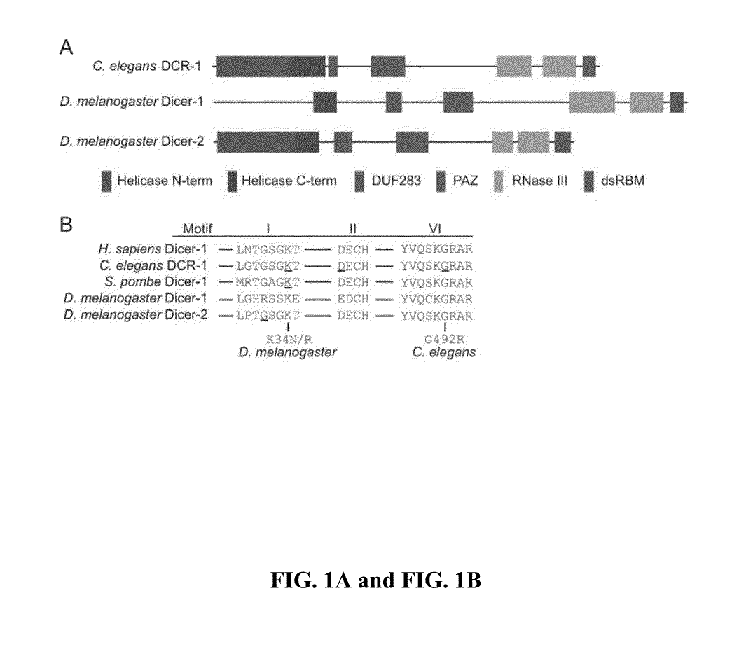

siRNAs and miRNAs are excised from double-stranded RNA (dsRNA) precursors by Dicer (See Hutvagner et al, Science 93, 834 (2001), Grishok at al., Cell 106, 23 (2001), and Bernstein et al., Nature 409, 363 (2001)), a multidomain RNase III protein, thus producing RNA species of similar size. Dicer endonucleases cleave long double-stranded RNA (dsRNA) and short hairpin RNA (pre-miRNA) into 20-30 nucleotide (nt) RNAs, called siRNAs and miRNAs, respectively. These short RNAs function as sequence-specific guides in targeting mRNAs for silencing. Most Dicer orthologs share a common domain architecture (see FIG. 1A). Biochemical and structural studies have provided detailed information about how the RNase III nuclease domains direct cleavage of dsRNA in the active site. The helicase domain contains conserved motifs that, in other Superfamily 2 helicases, couple ATP hydrolysis to motor activities such as unwinding or translocation (see FIG. 1B). A described herein, Caenorhabditis elegans strains expressing Dicer (DCR-1) with point mutations in either of three different helicase motifs (FIG. 1B, underlined) show normal levels of mature miRNAs, but are defective for the production of certain endogenous siRNAs (endo-siRNAs), particularly a longer 26 nt species with a 5′ guanosine (26G RNAs). While H. sapiens and C. elegans encode a single Dicer, D. melanogaster encodes two separate enzymes, one for processing miRNA precursors (Dicer-1) and the other for processing siRNAs from exogenously introduced, or endogenous, dsRNA (Dicer-2). Dicer-1 lacks a functional helicase domain (FIG. 1A) and does not require ATP for activity, further indicating pre-miRNA processing does not require a helicase function. In contrast, Dicer-2 has a well-conserved helicase domain and requires ATP to efficiently cleave long dsRNA. Consistent with the idea that the helicase domain of Dicer-2 is required for siRNA processing, a mutation in its Walker A motif (G31R, FIG. 1B) reduces siRNA derived from dsRNA produced from a transgene in vivo. Similarly, S. pombe strains expressing Dicer with a point mutation in the same helicase motif (K38A, FIG. 1B) are defective for centromeric silencing and generation of siRNAs.

Previous in vitro studies have not provided an obvious mechanistic function for the helicase domain of Dicer. In fact, deletion or mutation of the helicase domain of human Dicer leads to a more active enzyme in vitro. In continued pursuit of the mechanistic function of Dicer's helicase domain, in the present disclosure, in vitro studies using cell free-extracts of C. elegans, as well as purified recombinant Drosophila Dicer-2 are preformed. For both systems, activities of wildtype and mutant forms of the helicase domain of Dicer are compared.

One aspect of the present disclosure provides that the helicase domain of Dicer is required for distinguishing between different dsRNA termini. In another embodiment, the enzyme is engaged for processive cleavage of siRNA from internal regions of dsRNA. In yet another embodiment, the helicase domain is essential for in vivo accumulation of certain endo-siRNA, but not miRNA.

Disclosed herein are methods of producing siRNA in vitro and in vivo.

Disclosed herein are methods of producing siRNA in vitro comprising: introducing at least one dsRNA molecule and at least one Dicer enzyme comprising a helicase domain to a sample to form a reaction mixture, wherein the dsRNA comprises blunt ends or 5′ overhangs; and incubating the reaction mixture for a time sufficient to produce siRNA. In some aspects the Dicer enzyme is a Drosophila Dicer-2 enzyme, a C. elegans Dicer enzyme or a human Dicer enzyme. In some aspects, the siRNA produced comprises a 3′ overhang of 3 or 4 nucleotides. In some aspects, the dsRNA molecule of the reaction mixture does not comprise a 3′ overhang. In some aspects, the methods of producing siRNA in vitro further comprise introducing ATP to the reaction mixture.

In some embodiments, the methods of producing siRNA in vitro selectively produce siRNA without affecting the production of microRNA.

Also disclosed herein are methods of producing siRNA in vitro comprising: introducing at least one dsRNA molecule and at least one Dicer enzyme comprising a helicase domain to a sample to form a reaction mixture, wherein the dsRNA comprises blunt ends or 5′ overhangs; and incubating the reaction mixture for a time sufficient to produce siRNA, wherein the Dicer enzyme selectively binds to dsRNA comprising blunt ends or 5′ overhangs.

It is known that RNA interference (RNAi) is a mechanism by which double-stranded RNAs (dsRNAs) suppress specific transcripts in a sequence-dependent manner. dsRNAs can be processed by Dicer to about 19-30 nucleotide small interfering RNAs (siRNAs) and then incorporated into the argonaute (Ago) proteins. In mammals, where no RdRP activity has been found, biogenesis and function of endogenous siRNAs remain largely unknown. Studies in mice have suggested a role for endogenous siRNAs in mammalian oocytes and show that organisms lacking RdRP activity can produce functional endogenous siRNAs from naturally occurring dsRNAs (See for example, Wantanabe et al., Nature, 453(7194):539-43 (2008). As such, methods of modulating the production of siRNA in a subject has utility. Disclosed herein are methods of modulating the production of siRNA in a subject. In some aspects, production of siRNA in a subject is increased. In some aspects, production of siRNA in a subject is decreased.

Disclosed herein are methods of modulating the production of siRNA in a subject comprising: administering a mutated Dicer enzyme to the subject, wherein the Dicer enzyme comprises a mutation in the helicase domain of the Dicer enzyme, thereby modulating the production of siRNA in the subject. For example, disclosed herein are methods of modulating the production of siRNA in a subject comprising: administering a mutated Dicer enzyme to the subject, wherein the Dicer enzyme comprises a mutation in the helicase domain of the Dicer enzyme, thereby decreasing the production of siRNA in the subject

Disclosed herein are methods of modulating the production of siRNA in a subject comprising: administering a mutated Dicer enzyme to the subject, wherein the Dicer enzyme comprises a mutation in the helicase domain of the Dicer enzyme, thereby modulating the production of siRNA in the subject.

Also disclosed herein are methods of modulating the production of siRNA in a subject comprising: administering a Dicer enzyme comprising a helicase domain to the subject, thereby modulating the production of siRNA in the subject. For example, disclosed herein are methods of modulating the production of siRNA in a subject comprising: administering a Dicer enzyme comprising a helicase domain to the subject, thereby increasing the production of siRNA in the subject

Also disclosed herein are methods of modulating the production of siRNA in a subject comprising: administering a Dicer enzyme comprising a helicase domain to the subject, thereby modulating the production of siRNA in the subject, further comprising administering at least one dsRNA molecule comprising blunt ends or at least one 5′ overhang to the subject. For example, disclosed herein are methods of modulating the production of siRNA in a subject comprising: administering a Dicer enzyme comprising a helicase domain to the subject, thereby increasing the production of siRNA in the subject, further comprising administering at least one dsRNA molecule comprising blunt ends or at least one 5′ overhang to the subject. In some aspects, the further administration of at least one dsRNA molecule comprising blunt ends or at least one 5′ overhang to the subject further increases the production of siRNA in the subject when compared to solely administering a Dicer enzyme comprising a helicase domain to the subject.

The siRNA produced in the methods disclosed herein are also disclosed. For example, one aspect of the present disclosure therefore provides isolated siRNA comprising short double-stranded RNA from about 17 nucleotides to about 30 nucleotides in length, and in certain embodiments from about 19 to about 25 nucleotides in length, that are targeted to the target mRNA. The siRNA comprise a sense RNA strand and a complementary antisense RNA strand annealed together by standard Watson-Crick base-pairing interactions (hereinafter “base-paired”). As is described in more detail below, the sense strand comprises a nucleic acid sequence which is identical or closely homologous to a target sequence contained within the target mRNA.

The sense and antisense strands of the siRNA produced by the methods disclosed herein can comprise two complementary, single-stranded RNA molecules or can comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form an siRNA of two individual base-paired RNA molecules.

The siRNA can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA can also comprise a 3′ overhang. As used herein, a “3′ overhang” refers to at least one unpaired nucleotide extending from the 3′-end of a duplexed RNA strand. In a preferred embodiment, the “3′ overhang” refers to 3 or 4 unpaired nucleotide extending from the 3′-end of a duplexed RNA strand.

Thus, in one embodiment, the siRNA comprises at least one 3′ overhang of from 1 to about 6 nucleotides (which includes ribonucleotides or) in length, preferably from 1 to about 5 nucleotides in length, more preferably from 1 to about 4 nucleotides in length, and particularly preferably from about 3 to about 4 nucleotides in length.

Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon

The ability of an siRNA containing a given target sequence to cause RNAi-mediated degradation of the target mRNA can be evaluated using standard techniques for measuring the levels of RNA or protein in cells. For example, siRNA can be delivered to cultured cells, and the levels of target mRNA can be measured by Northern blot or dot blotting techniques, or by quantitative RT-PCR. Alternatively, the levels of the target protein in the cultured cells can be measured by ELISA or Western blot.

RNAi-mediated degradation of target mRNA by an siRNA containing a given target sequence can also be evaluated with animal models of particular disease.

RNAi-mediated degradation of the target mRNA can be detected by measuring levels of the target mRNA or protein in the cells of a subject, using standard techniques for isolating and quantifying mRNA or protein as described above.

One skilled in the art can readily determine an effective amount of the siRNA to be administered to a given subject, by taking into account factors such as the size and weight of the subject; the extent of the disease penetration; the age, health and sex of the subject; the route of administration; and whether the administration is regional or systemic. Generally, an effective amount of the siRNA comprises an intercellular concentration at or near the disease site of from about 1 nanomolar (nM) to about 100 nM, preferably from about 2 nM to about 50 nM, more preferably from about 2.5 nM to about 10 nM. It is contemplated that greater or lesser amounts of siRNA can be administered.

In the present methods, the present siRNA can be administered to the subject either as naked siRNA, in conjunction with a delivery reagent, or as a recombinant plasmid or viral vector which expresses the siRNA.

Suitable delivery reagents for administration in conjunction with the present siRNA include the Minis Transit TKO lipophilic reagent; lipofectin; lipofectamine; cellfectin; or polycations (e.g., polylysine), or liposomes. A preferred delivery reagent is a liposome.

Liposomes can aid in the delivery of the siRNA to a particular tissue, such as retinal or tumor tissue, and can also increase the blood half-life of the siRNA. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

Preferably, the liposomes encapsulating the present siRNA comprises a ligand molecule that can target the liposome to a particular cell or tissue at or near the target site. Particularly preferably, the liposomes encapsulating the present siRNA are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al. (1988), P.N.A.S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties can deliver the present siRNA to tumor cells.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM₁. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60° C.

The siRNA can be administered to the subject by any means suitable for delivering the siRNA to the cells of interest for treatment. For example, the siRNA can be administered by gene gun, electroporation, or by other suitable parenteral or enteral administration routes.

Suitable enteral administration routes include oral, rectal, or intranasal delivery.

Suitable parenteral administration routes include intravascular administration (e.g. intravenous bolus injection, intravenous infusion, intra-arterial bolus injection, intra-arterial infusion and catheter instillation into the vasculature); peri- and intra-tissue administration (e.g., peri-tumoral and intra-tumoral injection, intra-retinal injection or subretinal injection); subcutaneous injection or deposition including subcutaneous infusion (such as by osmotic pumps); direct (e.g., topical) application to the area at or near the site of neovascularization, for example by a catheter or other placement device (e.g., a corneal pellet or a suppository, eye-dropper, or an implant comprising a porous, non-porous, or gelatinous material); and inhalation.

The siRNA can be administered in a single dose or in multiple doses. Where the administration of the siRNA is by infusion, the infusion can be a single sustained dose or can be delivered by multiple infusions. Injection of the agent directly into the tissue is at or near the target site.

One skilled in the art can also readily determine an appropriate dosage regimen for administering the siRNA to a given subject. For example, the siRNA can be administered to the subject once, such as by a single injection or deposition at or near the target site. Alternatively, the siRNA can be administered to a subject multiple times daily or weekly. For example, the siRNA can be administered to a subject once weekly for a period of from about three to about twenty-eight weeks, and alternatively from about seven to about ten weeks. In a certain dosage regimen, the siRNA is injected at or near the target site once a week for seven weeks.

Where a dosage regimen comprises multiple administrations, it is understood that the effective amount of siRNA administered to the subject can comprise the total amount of siRNA administered over the entire dosage regimen.

The siRNA are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

In one embodiment, the pharmaceutical formulations comprise an siRNA (e.g., 0.1 to 90% by weight), or a physiologically acceptable salt thereof, mixed with a physiologically acceptable carrier medium. Preferred physiologically acceptable carrier media are water, buffered water, saline solutions (e.g., normal saline or balanced saline solutions such as Hank's or Earle's balanced salt solutions), 0.4% saline, 0.3% glycine, hyaluronic acid and the like.

Pharmaceutical compositions can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of one or more siRNA. A pharmaceutical composition for aerosol (inhalational) administration can comprise 0.01-20% by weight, preferably 1%-10% by weight, of one or more siRNA encapsulated in a liposome as described above, and propellant. A carrier can also be included as desired; e.g., lecithin for intranasal delivery.

Also disclosed herein are methods of screening for compositions that modulate siRNA production.

Described herein are methods of screening for a candidate modulator that modulates siRNA production comprising the steps of: (a) determining the production of siRNA in a sample comprising at least one dsRNA molecule and at least one Dicer enzyme comprising a helicase domain; (b) exposing the sample comprising at least one dsRNA molecule and at least one Dicer enzyme comprising a helicase domain to the candidate modulator; (c)

determining the effect of the candidate modulator on the production of siRNA in the sample; wherein a change in the production of siRNA in the sample after exposure to the candidate modulator is indicative of a modulator of siRNA production. In one aspect, the methods of screening for a candidate modulator that modulates siRNA production further comprise a high-throughput screen. In one aspect the present invention comprises compositions identified by the method of screening for a candidate modulator that modulates siRNA production.

As used herein, the term “diagnosed” means having been subjected to a clinical examination by a person of skill, for example, a physician, and found to have a condition that can be diagnosed or treated by the compounds, compositions, or methods disclosed herein. For example, “diagnosed with a disease caused by endogenous siRNA” means having been subjected to a clinical examination by a person of skill, for example, a physician utilizing the methods described herein, and found to have a condition that can be diagnosed as a disease caused by endogenous siRNA.

As used herein, “no increase” or “a decrease” means that there is no significant or perceptible increase in the number or concentration of endogenous siRNA when the subject is examined by a person of ordinary skill using molecular biology procedures well known in the art, such as the procedures described herein. Treatment can be in the form of administering one or more therapeutic agents to the subject alone or in combination with other forms of treatments including, but not limited to administering a Dicer enzyme comprising a helicase domain to the subject or administering at least one dsRNA molecule comprising blunt ends or at least one 5′ overhang to the subject.

Thus, a person of skill in the art can determine whether a course of treatment of a disease caused by endogenous siRNA in a subject is effective by following the subject at various time intervals and examining the genetic material of the subject to compare the number or concentration of endogenous siRNA at each examination to the number or concentration of endogenous siRNA determined at the initial examination when a disease caused by endogenous siRNA in the subject was first diagnosed.

Also disclosed herein are methods of treating a disease in a subject comprising: administering a mutated Dicer enzyme to the subject, wherein the Dicer enzyme comprises a mutation in the helicase domain of the Dicer enzyme, thereby treating the subject. In some aspects, the Dicer enzyme comprises a helicase domain wherein there is a mutation in the helicase domain that renders the Dicer enzyme partially or completely incapable of hybridizing to a dsRNA comprising blunt ends or a 5′ overhang. In some aspects, the mutation in the helicase domain can be a natural mutation or a synthetically derived mutation. In some aspects the disease can be caused by endogenous siRNA. In some aspects, the disease can be caused by overproduction of endogenous siRNA in the subject.

Also disclosed herein are methods of treating a disease in a subject comprising: administering a mutated Dicer enzyme and at least one dsRNA molecule comprising at least one 3′ overhang to the subject, wherein the Dicer enzyme comprises a mutation in the helicase domain of the Dicer enzyme, thereby treating the subject.

Also disclosed herein are methods of treating a disease in a subject administering a Dicer enzyme comprising a helicase domain to the subject, thereby treating the subject. In some embodiments methods of treating a disease in a subject further comprise administering a dsRNA molecule comprising blunt ends or at least one 5′ overhang to the subject. In some embodiments methods of treating a disease in a subject further comprise administering a dsRNA molecule comprising blunt ends or at least one 5′ overhang to the subject, wherein the dsRNA is specifically designed to comprise sequences capable of specifically hybridizing to a specific target nucleic acid. For example, the dsRNA can be designed to encode sequences that, when a Dicer enzyme comprising a helicase domain binds to the dsRNA, the dsRNA will be processed by the Dicer enzyme to produce siRNA molecules with 3′ overhangs that comprise a sequence targeted to and/or capable of specifically hybridizing to a specific target nucleic acid. In some aspects the disease can be caused by endogenous siRNA. In some aspects, the disease can be caused by underproduction of endogenous siRNA in the subject.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### Example 1

C. elegans Extract Cleavage Assay:

50 μl reactions containing extract cleavage buffer (30 mM HEPES pH 7.4, 100 mM KOAc, 10% glycerol, 1 mM DTT, 80 Units RNasin, 0.4 nM dsRNA, 4 mM ATP, and 6.5-12 mM MgOAc as indicated), were incubated at 20° C. with 40 μg C. elegans embryo extract (wildtype, N2; AH; dcr-1(mg375). Experiments used dcr-1(mg375) strain YY011, recently reported to contain a secondary mutation, mut-16(mg461); subsequent experiments with strain YY470, which lacks the mutation, gave identical results. Reactions were stopped by adding an equal volume of phenol/CHCl₃/Isoamyl alcohol (25:24:1) followed by organic extraction and ethanol precipitation after addition of 15 μg glycogen. RNAs were resolved by 17% denaturing PAGE and either exposed wet at −20° C. overnight on a PhosphorImager screen (³²P end-labeled dsRNAs), or subjected to northern blot analysis (“cold” dsRNAs). For northern blot analysis, nucleic acid was transferred from gels to Hybond-NX membrane (Amersham) in a wet transfer cell (80 V, 1 hour; 0.5×TBE). Blots were cross-linked with EDC (30 min; 60° C.; Pall et al., 2007), and incubated with 3 pmol of ³²P-end-labeled DNA probe at 42° C. in ULTRAhyb-Oligo buffer (Ambion). Membranes were washed 3-4 times at 42° C. in 2×-4×SSC+0.1-0.2% SDS, and exposed on a PhosphorImager screen (Molecular Dynamics). Between probings, blots were stripped by rotating at 80° C. with 20 mM Tris, pH. 7.5, 1 mM EDTA, 1% SDS, 3-4 times over 1.5 hours, and exposed to ensure all radioactivity was removed. Data were quantified using ImageQuant software. Alkaline Hydrolysis ladder was prepared by incubating 20 fmol of ³²P-end labeled dsRNA and 10 μg of non-specific (torula) RNA in 10 μl 50 mM sodium carbonate (pH 9.0, NaHCO₃/Na₂Co₃) at 85° C. for 10 min.

dsRNA Substrates and Probes:

40/42 and 106 RNA strands were chemically synthesized and gel-purified after 8% denaturing PAGE. For ³²P-end-labeled RNAs, the indicated RNA strands were labeled using T4 polynucleotide kinase, followed by gel purification after 8% denaturing PAGE. Equimolar amounts of single-stranded RNAs were annealed by heating (95° C., 1-5 min) in annealing buffer (10 mM Tris pH 7.5, 40 mM KCI) and slow cooling. dsRNAs were gel-purified after 6% native PAGE. ³²P-104 bp and 300 bp cold dsRNAs were prepared by in vitro transcription in the presence or absence of ³²P-ATP, followed by hybridization and gel-purification, annealing was for 5′ at 95° C. followed by slow cooling at room temp for more than 45 min.

Drosophila Dicer-2 Cloning and Protein Expression:

Wildtype and mutant Dicer-2 were cloned, overexpressed and purified as described (Ye and Liu, 2008). Briefly, Dicer-2 cDNA was cloned by reverse transcription of total RNA from Drosophila S2 cells using the RLM-RACE kit (Ambion), and K34N and K34R mutations were introduced using the Quickchange kit (Stratagene). Using PCR, sequences for poly-histidine tags were inserted at the N and C termini of all Dicer-2 open reading frames and cDNAs were cloned into pFastBac vector. The resulting plasmids were used to transform E. coli DHIOBac competent cells to make recombinant Bacmids. Recombinant proteins were expressed in Sf21 insect cells in suspension culture (27° C.). Cells were lysed and recombinant proteins purified using Ni-affinity column followed by SP- and Q-Sepharose chromatography. All purified proteins were estimated to be ≧90% pure.

Dicer-2 Cleavage Assay:

Wildtype and mutant Dicer-2 were cloned, over-expressed and purified as described above. Dicer-2 cleavage assays were performed at 24° C. for times indicated in cleavage buffer (30 mM HEPES (pH 7.4), 100 mM KOAc, 10 mM MgOAc, 1 mM DTT, 50 Units RNasin) containing Dicer-2 and dsRNA as specified. Protein and RNA were preincubated (15 min, 24° C.) and reactions started by adding ATP-MgOAc (5 mM final), unless otherwise specified. Reaction of 40/42 nt dsRNAs were performed by adding ATP-MgOAc (8 mM final) to cleavage mixture and starting reactions by adding protein.

Dicer-2 cleavage reactions were stopped by adding an equal volume of 2× formamide loading buffer (85% formamide, 0.5×TBE, 50 mM EDTA, pH 8.0, 0.05% bromophenol blue and 0.05% xylene cyanol). Reaction products were separated by 12% denaturing PAGE unless otherwise indicated, visualized on a PhosphorImager screen (Molecular Dynamics), and quantified using ImageQuant software. Northern blot assays were performed identically to those described for C. elegans in vitro cleavage assays.

### Example 2

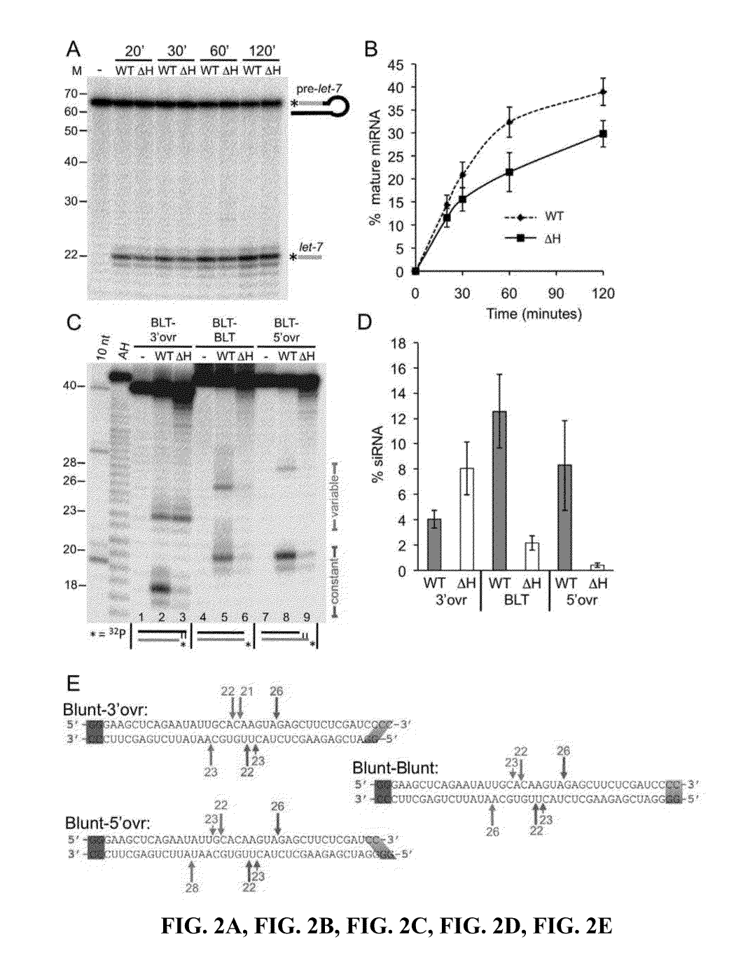

The domain of C. elegans Dicer mediates differential recognition of duplex termini: To gain mechanistic insight into the role of Dicer's helicase domain, processing of dsRNA by cell-free embryo extracts of wild-type (WT) C. elegans, or strains harboring a point mutation in motif VI (G492R, FIG. 1B) of the helicase domain (AH; dcr-1(mg375)) were compared. First, the reaction of a ³²P-end-labeled pre-let-7 RNA that matched the endogenous C. elegans sequence, including the characteristic 2 nt 3′ overhang were monitored. Both extracts processed pre-let-7, showing a similar accumulation of 22 nt mature let-7 over time (FIGS. 2A and 2B).

Precursors of endo-siRNA in D. melanogaster can arise from long, genomically-encoded hairpins, or overlapping genes that give rise to complementary transcripts. In addition, in vitro studies of human Dicer show that the length of an siRNA depends on the termini of its dsRNA precursor. Thus, as a first step in evaluating the requirement of Dicer's helicase domain in processing siRNA precursors, 40 and 42 nt RNAs were hybridized to create completely base-paired dsRNA with a variety of termini (FIGS. 2C and 2E). All dsRNAs had one blunt terminus and one terminus that varied to include a 2 nt 3′ overhang (BLT-3′ovr), a second blunt end (BLT-BLT), or a 2 nt 5′ overhang (BLT-5′ovr).

In choosing cleavage sites, Dicer measures from duplex termini, and the short length of the 40/42 dsRNAs allowed only one cleavage event, which could occur from either end. 40/42 dsRNAs were 5′ ³²P-end-labeled on one strand and designed so that cleavage resulting from measuring from the constant blunt terminus gave shorter products (˜18-20 nt, constant) than cleavage from the variable terminus (23-28 nt, variable; FIG. 2C). Each substrate was incubated with WT or ΔH extract and reaction products resolved by electrophoresis on a denaturing gel. In WT extracts, cleavage products were observed from all three variable termini and the constant blunt terminus (FIGS. 2C, lanes 2, 5, 8 and 2D). In contrast, while cleavage from the variable 3′ 2 nt overhang was observed in the AH extract (FIG. 2C, lane 3), these extracts were markedly deficient in processing from blunt termini or those with 5′ 2 nt overhangs (FIGS. 2C, lanes 3, 6, 9 and 2D). These data indicated that Dicer requires a functional helicase domain for efficient cleavage of dsRNA with blunt or 5′ overhanging termini, but not dsRNA with 3′ overhanging termini. The latter is consistent with the observation that Dicer's helicase domain is not required for processing pre-miRNA, which have 2 nt 3′ overhangs.

### Example 3

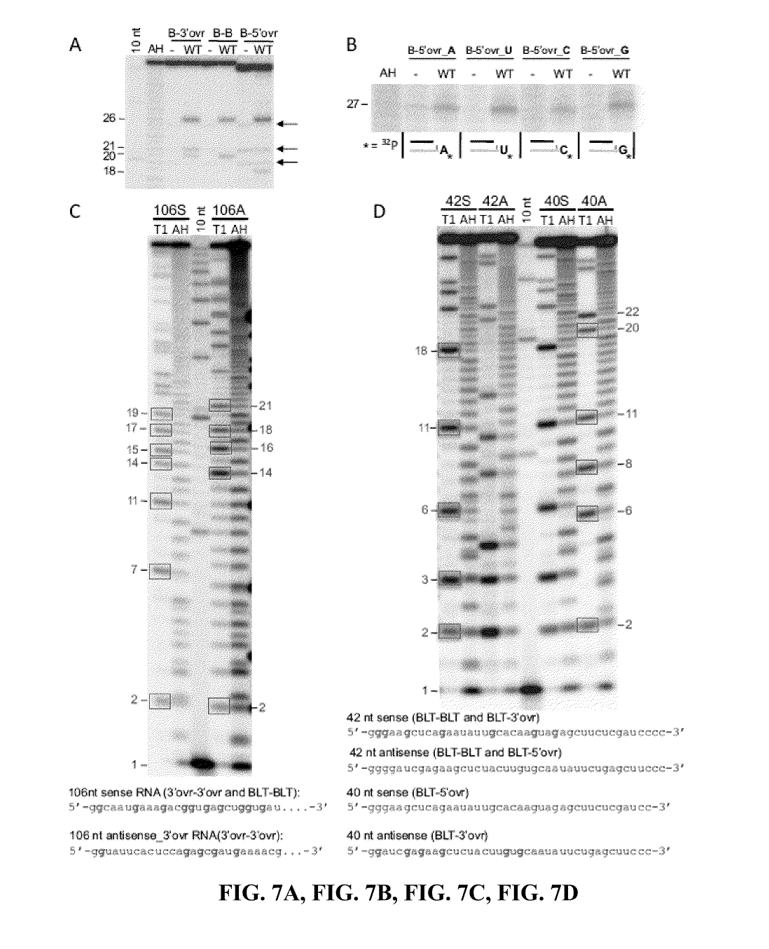

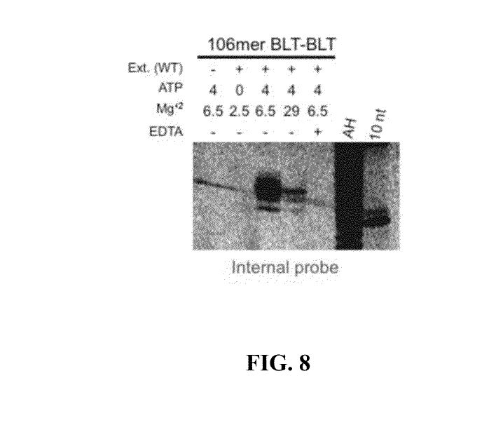

Dicer activity in C. elegans extracts produces siRNA with 3-4 nucleotide overhangs and lengths dependent on dsRNA termini: To more precisely define the cleavages occurring in the C. elegans extracts, reactions in WT extracts using 40/42 dsRNAs that were ³²P-end-labeled on the opposite strand were performed (FIG. 2C, black strand in cartoon, and 7). The migration of alkaline hydrolysis and T1 ribonuclease products were compared with 10 nt RNA ladders on long sequencing gels to obtain accurate sizes of cleavage products (FIG. 7). When combined, data for each strand allowed precise characterization of cleavage events (FIG. 2E). Each arrow of FIG. 2E indicates a site of cleavage that splits the molecule into a 5′ and 3′ cleavage product. While the length of 5′ cleavage products varied between molecules, 3′ cleavage products were of similar length, indicating that C. elegans Dicer measures from 3′ termini. However, 3′ cleavage products were 21 or 22 nts when measuring from 3′ overhanging termini, but 1 nt longer (22-23 nts) when measured from blunt or 5′ overhanging termini (FIG. 2E). Given that a functional helicase domain was required for efficient cleavage of dsRNA with blunt and 5′ overhanging termini, but not dsRNA with 3′ overhanging termini (FIG. 2C), these data suggest Dicer uses an alternate mode of cleavage when the helicase domain is engaged. It was also demonstrated that product lengths were consistent with a staggered cleavage of each strand to yield 3 and 4 nt overhangs, rather than the canonical 2 nt overhang associated with other RNase III family members. Deep sequencing of C. elegans small RNAs reveal 26 nt endo-siRNAs that pair with their sense partner strands in a manner that predicts a 3 nt 3′ overhang. This data demonstrates that 26 nt endo-siRNAs arise from a helicase-dependent cleavage from a blunt terminus (FIG. 2E). Most C. elegans 26 nt endo-siRNAs have a guanosine at their 5′ terminus. It is further demonstrated that duplexes with different 5′ nts were cleaved similarly by C. elegans extracts (FIG. 7), suggesting this feature does not derive from Dicer.

### Example 4

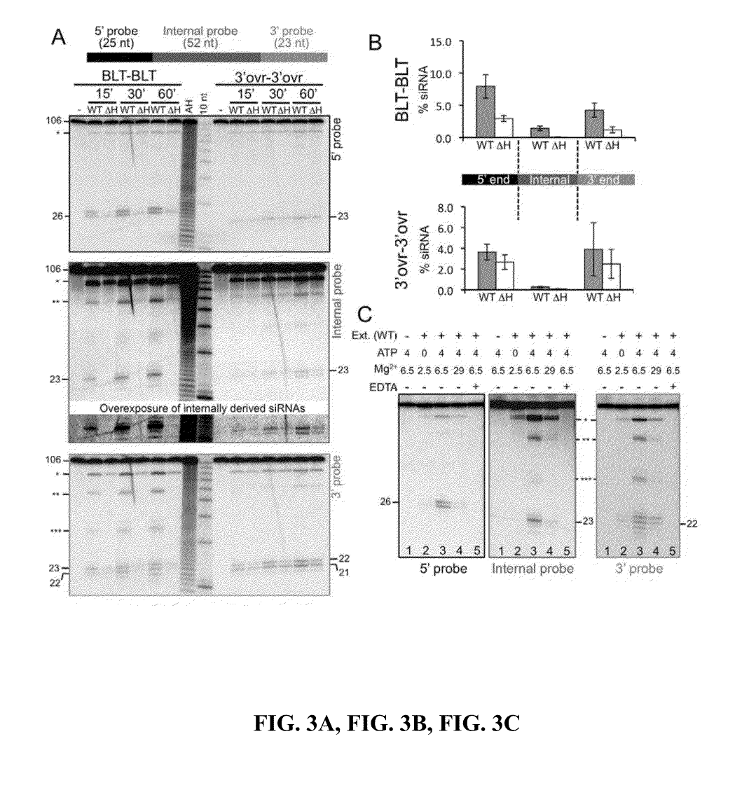

Dicer's helicase domain facilitates production of siRNAs from internal regions of long dsRNA with blunt, but not 3′ overhanging, termini: Many DEXH helicases act as translocases that couple ATP hydrolysis to movement along a nucleic acid. In fact, the ATP-dependence of Drosophila Dicer-2 is proposed to reflect a role of the helicase domain in translocation along a dsRNA substrate. The possibility that blunt and 5′ overhanging termini, but not 3′ overhangs, engaged the helicase domain for translocation and processive cleavage (i.e. one binding event followed by multiple cleavage events before dsRNA release) was considered. It was reasoned that if this model were true, the abundance of siRNAs from internal regions of a long dsRNA would be affected by the presence or absence of a functional helicase domain, as well as the terminus of a dsRNA. To test this hypothesis, cleavage reactions using nonradioactive dsRNA formed by hybridizing 106 nt RNAs were performed and reaction products were analyzed by northern blot using probes that hybridized to siRNAs from duplex termini, or alternatively, to those from internal regions (FIG. 3).

Representative blots for three time-points of a reaction with a BLT-BLT or a 3′ovr-3′ovr dsRNA incubated in a WT or AH extract are shown (FIG. 3A), and average values from multiple analyses plotted (FIG. 3B). When probed for siRNAs resulting from the first cleavage event at either end (top and bottom panels, 5′ probe and 3′ probe respectively), the sizes of predominant siRNA bands were reminiscent of those observed with 40/42 dsRNA. For example, the predominant 3′ cleavage products (3′ probe) from the 106 BLT-BLT dsRNA (22/23 nts) were 1 nt longer than those from the 106 3′ovr-3′ovr dsRNA (21/22). Similarly, a 5′ terminal siRNA (5′ probe) of 26 nt was observed for the 106 BLT-BLT dsRNA, and one of 23 nt for the 106 3′ovr-3′ovr dsRNA, as observed for 40/42 dsRNAs with similar termini (compare to FIGS. 2C and 2E). However, in addition to the 26 nt siRNA from the 5′ terminus of the 106 BLT-BLT dsRNA, a 27 nt band was also observed, after incubation in WT but not AH extracts. Since this helicase-dependent siRNA was observed with 106 dsRNAs, but not 40/42 dsRNAs, possibly it relates to processing of longer dsRNA. Analysis of blots with internal probes allowed visualization of subsequent cleavage events on the longer molecules, and interestingly, siRNAs from subsequent cleavage events were predominantly 23 nt in length, regardless of which strand was probed (FIG. 3A, middle panel; data not shown)

The 40/42 dsRNAs only allow a single cleavage event, measured from either end, and this was most analogous to the first cleavage event from the termini of the 106 dsRNAs, detected by the 5′ and 3′ probes. As observed for 40/42 dsRNAs (FIGS. 2C and 2D), WT and AH extracts produced similar levels of terminal siRNAs from the 3′ovr-3′ovr dsRNA, but the ΔH extracts were much less efficient in processing the BLT-BLT dsRNA compared to the WT extracts (FIG. 3A, 5′ and 3′ probes), and this was validated in multiple analyses (FIG. 3B). However, siRNAs from both 5′ and 3′ ends were detectable after incubation of the 106 BLT-BLT dsRNA in the AH extract, while those from internal regions were undetectabie, even after overexposure (FIGS. 3A and 3B). This result was consistent with the idea that, after Dicer recognizes a blunt terminus, a functional helicase domain is required for translocation and processive cleavage of internal regions of the duplex. Low levels of internal siRNAs were also detected after incubation of the 3′ovr-3′ovr molecule in the AH extract, possibly resulting from distributive cleavage, whereby Dicer dissociates from dsRNA after each cleavage event, and subsequent cleavage events occur only by rebinding. The latter would be inefficient, and consistent with this idea, when incubated with WT extract, higher levels of internal siRNAs were observed for the BLT-BLT dsRNA than the 3′ovr-3′ovr dsRNA (FIG. 3B). The complete absence of internal fragments when the 106 BLT-BLT dsRNA was incubated with ΔH extract suggests that this extract cannot cleave this dsRNA, even distributively. Possibly in the absence of a functional helicase domain, Dicer inefficiently dissociates from blunt termini.

Another distinctive feature of cleavage of long dsRNA in the C. elegans extracts was the appearance of higher molecular weight reaction intermediates corresponding to one, two or three cleavage events (asterisks; FIG. 3A), predominantly with the BLT-BLT molecule. Such intermediates were observed with the internal probes, as well as with the 5′ and 3′ probes, since the latter could detect siRNAs from terminal regions, but also intermediates corresponding to successive cleavage events from the opposite end. In theory, under optimal conditions, a processive enzyme could catalyze rapid and successive cleavage events without the accumulation of intermediates. The observation of intermediates was consistent either with a very slow processive cleavage or a distributive cleavage. The former possibility is favored since the reaction in an extract is likely suboptimal, and further, intermediates were most abundant for the BLT-BLT dsRNA incubated in WT extracts, indicating that a functional helicase domain was required. Furthermore, intermediates were more readily observed with the internal and 3′ probes, compared to the 5′ probe.

### Example 5

In C. elegans extracts the accumulation of siRNAs from internal regions of dsRNA is ATP-dependent: Motor activities associated with characterized DEXH helicases are coupled to ATP-hydrolysis. It was reasoned that cleavage events that were helicase-dependent should also be dependent on ATP. Indeed, without the addition of ATP, reaction of 106 BLT-BLT dsRNA showed a small amount of siRNA from termini but none from internal regions, as observed in AH extracts (FIG. 3C, lane 2 all panels), even after overexposure (FIG. 8). Similarly, the helicase-dependent 27 nt siRNA observed with the 5′ probe was also ATP-dependent (compare lanes 2 and 3, 5′ probe). Further, addition of ATP enhanced siRNA accumulation from internal regions of the 106 Blunt-Blunt dsRNA (compare lane 2 and 3, internal probe), and also gave rise to intermediates (lane 3, internal and 3′ probe). As expected from Dicer's known dependence on divalent, magnesium was necessary for cleavage (lane 5, all panels), albeit at high concentrations (about 25 mM free Mg²+), inhibited the accumulation of reaction products (lane 4, all panels).

### Example 6

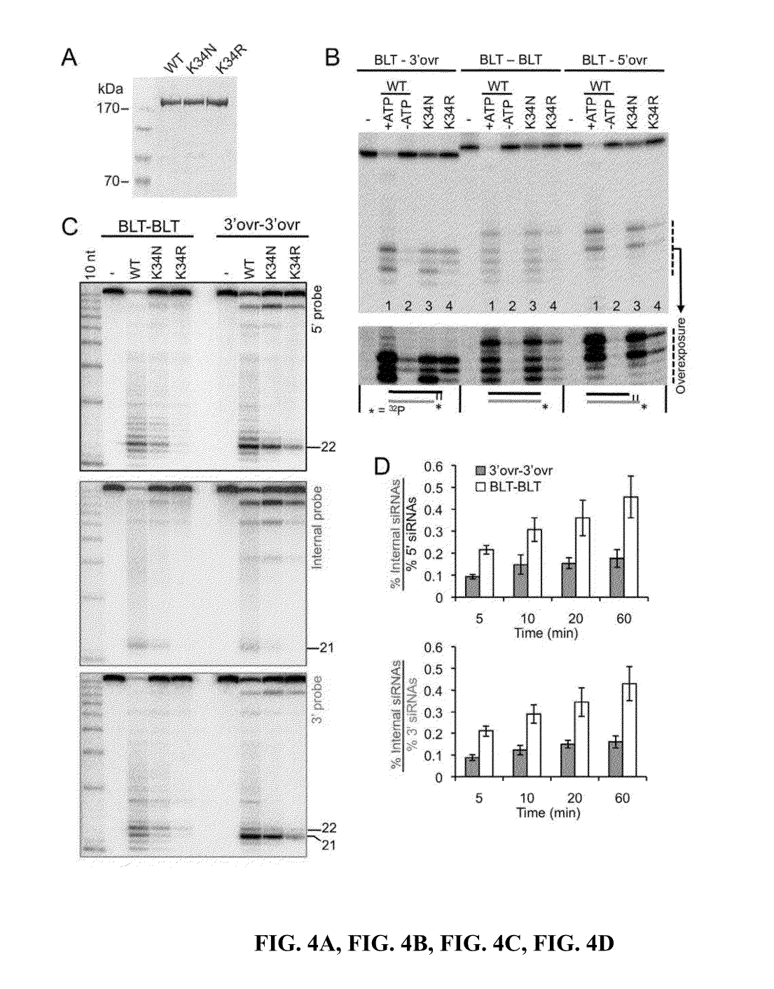

Purified Drosophila Dicer-2 also discriminates duplex termini: Comparisons of cleavage in WT and AH C. elegans extracts indicated that the helicase domain was required for recognition of duplex termini and the efficient accumulation of siRNAs from internal regions of dsRNA. However, because extracts containing many proteins were used, it was not revealed whether helicase-dependent activities were intrinsic to Dicer, or mediated in concert with other proteins. To address this question, studies of Drosophila Dicer-2 were initiated, which has a well conserved helicase domain (FIG. 1A) and like C. elegans DCR-1, is implicated in endo-siRNA processing. The WT Dicer-2 was over-expressed and purified, as well as variants with mutations in the Walker A motif of the helicase domain (FIGS. 1B and 4A).

The cleavage of the 40/42 dsRNAs was monitored and cleavage by Drosophila Dicer-2 was very dependent on the addition of ATP (FIG. 4B, compare lanes 1 and 2 for all dsRNAs). However, in the absence of ATP, cleavage was virtually undetectable for the BLT-BLT and BLT-5′ovr 40/42 dsRNAs, but a small but reproducibly detectable amount of cleavage was observed for the BLT-3′ovr dsRNA (FIG. 4B, compare lane 2 for all dsRNAs). Thus, purified Drosophila Dicer-2 reacted differently on dsRNA with 3′ overhanging termini compared to those with blunt and 5′ overhanging termini, providing the first hint that the properties observed in C. elegans extracts were intrinsic to Dicer.

The residue that was mutated in the Drosophila Dicer-2 helicase domain (K34) is well studied in other helicases, and in some cases eliminates helicase function. However, relatively conservative substitutions were chosen, and found that when reacted with the 40/42 dsRNAs, the K34N mutant showed nearly wildtype levels of cleavage (FIG. 4B, compare lanes 1 and 3 for all dsRNAs). In contrast, the K34R mutant, like the G492R helicase mutant of C. elegans, was able to process 3′ overhanging termini but was very inefficient in processing blunt and 5′ overhanging termini (FIG. 4B, lane 4 for all dsRNAs).

In addition, the reaction of non-radiolabeled 106 dsRNAs with wildtype and helicase mutant forms of Drosophila Dicer-2 were monitored, and northern blot assays using probes for terminal and internal siRNAs were performed (FIG. 4C). In agreement with results using the 40/42 dsRNAs, terminal siRNAs were readily detected with wildtype and both mutant proteins when reacted with the 3′ovr-3′ovr 106 dsRNA, but only wildtype Dicer-2. The K34N mutant showed efficient production of terminal siRNAs with BLT-BLT 106 dsRNA. Internal siRNAs were most abundant for the reaction of BLT-BLT 106 dsRNA with wildtype Dicer-2, and quantification of multiple time-course experiments with this enzyme emphasized that processing of 106 Blunt-Blunt molecules gave rise to many more internal siRNAs relative to terminal siRNAs than processing of 106 3′ovr-3′ovr dsRNAs (FIG. 4D). Again, the K34R mutant behaved similarly to the AH C. elegans mutant (G492R), and internal siRNAs were undetectable with this protein. The reactions of FIG. 4C were performed at 10-fold excess Dicer-2 compared to dsRNA, and with this condition intermediates were most prominent for the 3′ovr-3′ovr dsRNA, consistent with the idea that this molecule reacts distributively.

### Example 7

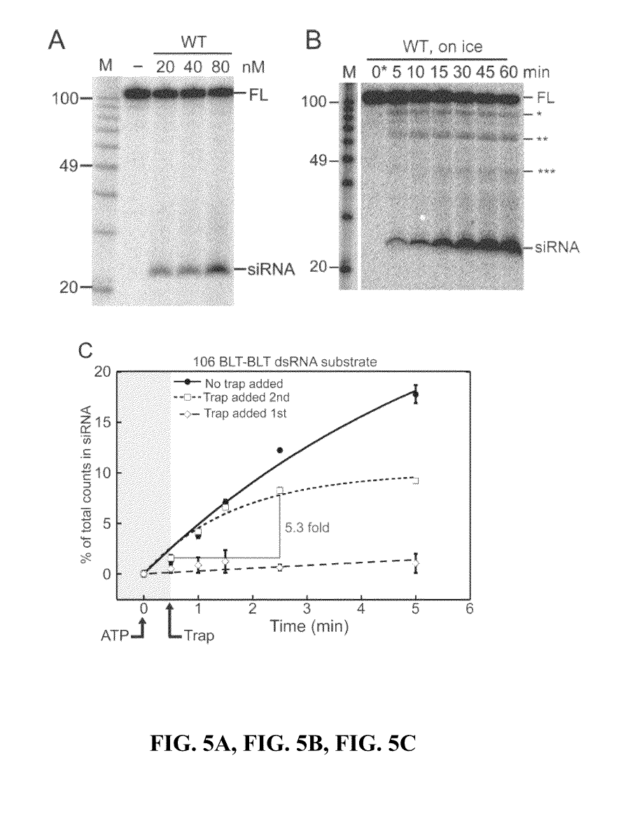

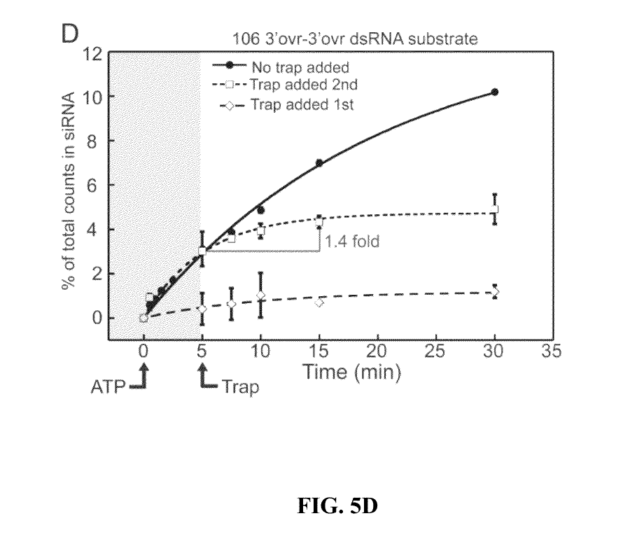

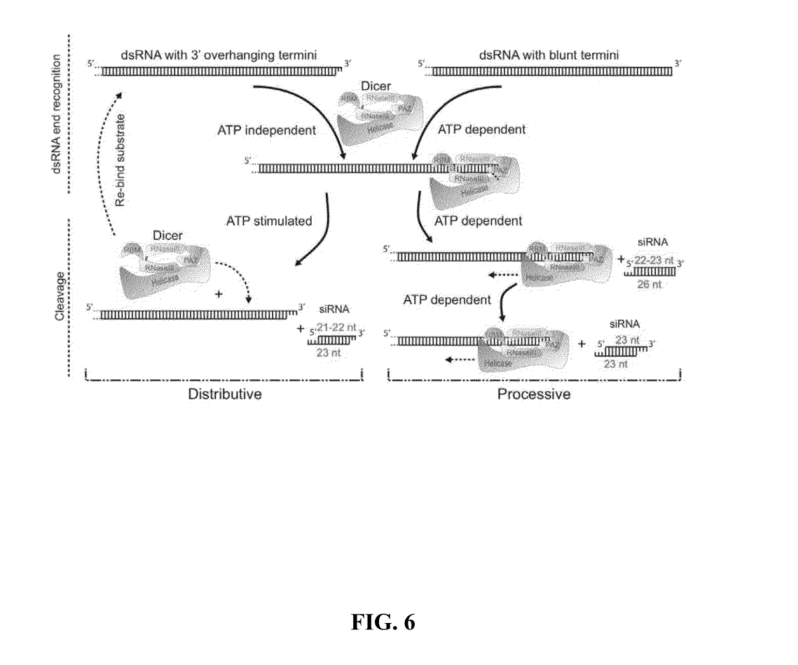

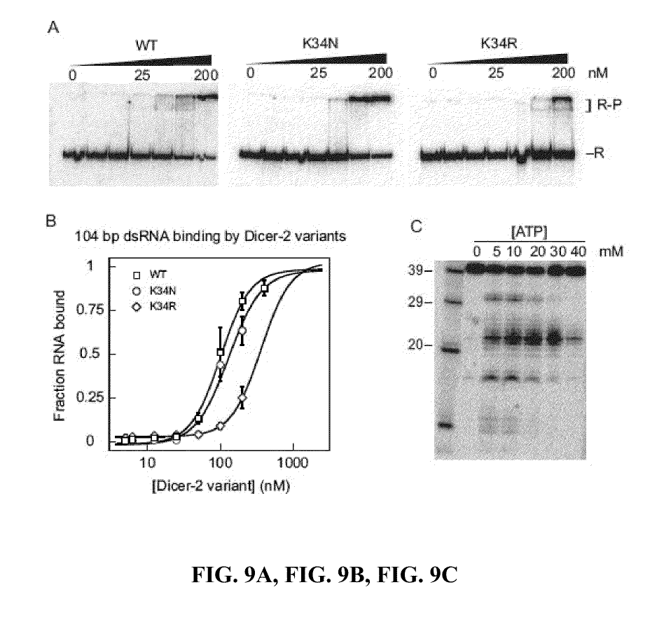

Drosophila Dicer-2 switches from a processive to a distributive reaction at high concentrations of ATP: Data from studies of DCR-1 in C. elegans extracts, and purified Drosophila Dicer-2, suggested a model whereby Dicer's helicase domain engages blunt and 5′ overhanging termini for translocation, enabling processive cleavage of long dsRNA. To gain further evidence for this model, several assays using the purified, recombinant Dicer-2 were performed. To facilitate analyses, a 104 bp dsRNA with a 1 nt 5′ overhang that was internally labeled with ³²P was prepared (³²P-104 bp (dsRNA-FL, FIG. 5A). Further, to maximize differences that might be related to processivity, reactions with excess Dicer-2 were performed to approximate single-turnover conditions. Using these conditions, WT Dicer-2 cleaved 32P-104 bp dsRNA efficiently into siRNAs, while siRNA products were barely detected (K34N) or undetectable (K34R) with the mutant proteins. Given the earlier results (FIG. 4), the small amount of siRNAs observed with the K34N mutant likely arises from cleavage from termini and not internal cleavage events. While the mutant proteins were defective in dsRNA cleavage, they bound dsRNA with affinities within an order of magnitude to that of WT Dicer-2, albeit all proteins bound weakly to ³²P P-104 bp dsRNA (WT K_{D}=101±7 nM; FIG. 9).

Given the sensitivity of Dicer-2 activity to the presence or absence of ATP, the optimal ATP concentration for the cleavage reaction were sought (FIG. 5B). Reactions with increasing concentrations of ATP were performed, in each case adding an equivalent concentration of magnesium to maintain a constant amount of free divalent cation. Unexpectedly, the cleavage of the ³²P-104 bp dsRNA by Dicer-2 was markedly different depending on ATP concentration. At ATP concentrations ≦10 mM, the predominant products were siRNAs. However, as ATP concentration was increased, the levels of siRNA decreased, and higher molecular weight cleavage intermediates appeared, similar to those observed for the 3′ovr-3′ovr 106 dsRNA at lower ATP concentrations (5 mM, FIG. 4C).

To provide further evidence that blunt termini triggered a reaction whereby multiple siRNAs along the length of the dsRNA were produced without Dicer-2 dissociation, a “pulse-chase” approach was used. Dicer-2 was allowed to cleave ³²P internally-labeled dsRNA for a short time to allow Dicer-2-dsRNA complex formation, followed by the addition of a vast excess of nonradiolabeled (“cold”) dsRNA trap. If Dicer-2 cleaved distributively, dissociating and rebinding after each cleavage event, addition of trap would quench the reaction and siRNAs should fail to accumulate after trap addition. In contrast, siRNAs would continue to accumulate even after the addition of trap if multiple siRNAs could be produced along the length of a dsRNA without Dicer-2 dissociation.

The reaction of internally labeled 32P-106 BLT-BLT or 32P-106 3′ovr-3′ovr dsRNA in the absence of all trap was monitored (FIG. 5C, D, filled circles, solid line) and also confirmed that 2000 fold excess of cold trap (82 BLT-BLT) over 32P-labeled dsRNA was sufficient to quench the reaction if added at the beginning of the reaction (diamonds, dashed line). Each was then reacted with ³²P-dsRNA for a short time (FIG. 5C, D, shaded), followed by the addition of trap dsRNA. Quantification of multiple experiments revealed that even after the addition of trap, siRNAs continued to accumulate from ³²P-106 BLT-BLT dsRNA (FIG. 5C), increasing 5.3 fold in the 2 minutes following trap addition. In contrast, siRNAs from 3′ovr-3′ovr dsRNA increased only 1.4 fold in the 10 minutes following trap addition; this small increase may be due to the final cleavage event for Dicer-2 productively bound to dsRNA immediately prior to the trap addition. While multiple siRNAs were produced without Dicer-2 dissociation from the 32P-106 BLT-BLT dsRNA, siRNAs did not reach the same maximum after trap addition as in the complete absence of trap, suggesting a small amount of dissociation occurred from all reacting complexes, or alternatively, that a small subset of complexes were not resistant to trap.

A classic test of processivity involves a “trap experiment”, which in this system would require formation of a stable dsRNA-Dicer complex in the absence of cleavage, followed by initiation of cleavage and the simultaneous addition of a large excess of a dsRNA “trap”. Thus, as a further test of processivity, reactions at low ATP concentrations were performed with a constant amount of ³²P-104 bp dsRNA, and increasing amounts of a nonradioactive dsRNA (4-4000 excess compared to labeled dsRNA; 0.025-25 fold excess compared to protein). If Dicer was reacting distributively at low ATP, dissociating and rebinding after each cleavage event, an increase in cleavage intermediates is expected with increasing amounts of non-radiolabeled dsRNA. In contrast, if Dicer were acting processively, each binding event would result in cleavage of the entire dsRNA into multiple siRNAs before dissociation, and intermediates would not appear. The latter was observed, and intermediates were not observed under any conditions.

The activity of wildtype Dicer and Dicer containing point mutations in its helicase domain were compared, using C. elegans extracts or purified recombinant Drosophila Dicer-2. In both systems, it was found that the helicase domain allows Dicer to discriminate between dsRNA with different termini. It was demonstrated that the helicase domain is required for efficient cleavage of dsRNA with blunt or 5′ overhanging termini, but not dsRNA with 3′ overhanging termini. The latter agrees with previous in vivo studies that indicate the helicase domain is required for processing certain endo-siRNAs, but not miRNAs, which have a 3′ overhanging terminus.

Using long dsRNAs that can accommodate multiple cleavage events, it was observed that the helicase domain also facilitates production of siRNAs from internal regions of dsRNA, for molecules with blunt, but not 3′ overhanging termini. Taken together this data suggests a model whereby dsRNAs with blunt or 5′ overhanging termini engage the helicase domain of Dicer for translocation along dsRNA and processive cleavage.

Model for helicase-dependent, processive cleavage of dsRNA by Dicer: FIG. 6 illustrates a model for the function of Dicer's helicase domain. Given the properties of the helicase mutants and the observed ATP-requirements, the reaction is described in two steps: 1) dsRNA end recognition and 2) cleavage. Binding of the PAZ domain to the 3′ terminus of dsRNA is crucial for orienting the RNase III domains for cleavage and this model shows this as the first step, regardless of the terminal structure of dsRNA. However, as illustrated, this would be an ATP-independent step for dsRNA with 3′ overhangs, but for blunt termini would require ATP to enable the helicase domain to unwind termini, allowing the PAZ domain access to the 3′ end. Assuming ATP-dependence equates to a dependence on the helicase domain, this aspect of the model is consistent with the observation that C. elegans AH extracts, or the Drosophila K34R mutant, are defective for cleavage of dsRNA with blunt or 5′ overhangs but show wildtype levels of terminal siRNA from dsRNA with 3′ overhangs (FIGS. 2C and 2D; FIGS. 3A and 3B; FIGS. 4B and 4C).

Depending on duplex termini, end-recognition can be ATP-independent or ATP-dependent, but in this model the consequence is the same: interaction of the PAZ domain with the 3′ terminus. However, in the present model, subsequent steps differ depending on duplex termini, or in the case of purified Drosophila Dicer-2, the ATP concentration. With duplexes containing 3′ overhanging termini, or at high ATP, Dicer acts distributively, dissociating from the dsRNA after each cleavage event, followed by rebinding and another cleavage. This aspect of the model is supported by the observation that production of siRNAs from internal regions of a dsRNA with 3′ overhangs is inefficient (FIGS. 3B and 4D), and in some cases, intermediates that are consistent with a distributive reaction are observed (FIGS. 4C, 5C and 5D).

While a small amount of cleavage from 3′ overhanging termini was observed in the absence of ATP (FIG. 4B, lane 2), it is shown the distributive cleavage step as ATP-stimulated (FIG. 6), since addition of ATP increased siRNA production by wildtype Drosophila Dicer-2 (FIG. 4B, lane 1). ATP also stimulated cleavage by both Dicer-2 helicase mutants (FIG. 4B, lanes 3 and 4), indicating that, at the least, these mutants can bind ATP. ATP binding or hydrolysis could be important for turnover, or as observed in other helicases, a protein conformational change, in this case to stabilize Dicer binding to duplex termini. Importantly, since pre-miRNA substrates, which possess 2 nt 3′ overhanging termini, require only one cleavage event to produce a mature miRNA, they are perfectly suited for distributive cleavage.

The model proposes that cleavage of dsRNA with blunt or 5′ overhanging termini is processive, with Dicer catalyzing multiple cleavage events before dissociation. This aspect of the model is supported by the observation that the helicase domain is required for (FIGS. 3A and 3B) or stimulates (FIGS. 4C and 4D) the accumulation of siRNAs from internal regions of dsRNA with blunt termini, which is completely dependent on ATP (FIG. 3C).

Further, with purified Drosophila Dicer-2, siRNAs accumulate without the appearance of intermediates (FIG. 5A). Finally, during trap experiments siRNAs continue to accumulate from BLT-BLT dsRNAs even after the addition of 2000 fold excess trap, indicating that multiple siRNAs are produced without Dicer-2 dissociation (FIG. 5C). Many DEXH helicases act as translocases that couple ATP hydrolysis to movement along a nucleic acid (Lohman et al., 2008; Singleton et al., 2007). In fact, the ATP-dependence of Drosophila Dicer-2 is proposed to reflect a role of the helicase domain in translocation along dsRNA (Bernstein et al., 2001; Hutvagner and Zamore, 2002; Nykanen et al., 2001) Dicer's PAZ domain acts as part of a ruler that “measures” from the 3′ end of dsRNA to specify the cleavage site. Using C. elegans extracts, it was observed that duplexes with 3′ overhanging termini were cleaved at 21-22 nts from the 3′ terminus, while dsRNA with blunt termini were cleaved at 22-23 nts from the 3′ terminus. Unexpectedly, C. elegans cleavage products from both blunt and 3′ overhanging dsRNAs showed non-canonical 3 and 4 nt 3′ overhangs. Here it is important to note that after cleavage to remove the first siRNA from the end of a dsRNA, all dsRNAs have 3′ overhangs, and subsequent cleavage events yield 23 nt siRNAs for all dsRNAs. Despite this, subsequent cleavage of dsRNA with blunt and 3′ overhangs is very different, emphasizing that the former likely involves a processive reaction whereby the enzyme remains bound for multiple cleavage events.

Previous studies in C. elegans indicate that miRNAs represent about 80-90% of all small RNAs, with primary endo-siRNAs accounting for only a small fraction, about 1%. While endo-siRNAs precursors are ill-defined, they are likely of low abundance, and processive cleavage would be an efficient way to maximize endo-siRNA levels.

Implications for the role of Dicer's helicase domain in processing small RNAs of other organisms: Precursors of miRNAs have been analyzed in many organisms, and all are assumed to have 2 nt 3′ overhangs. The present data suggest processing of these small RNAs will not require Dicer's helicase domain. This is supported by the existence of a second Dicer in D. melanogaster (Dicer-1), which is dedicated to miRNA processing and lacks a functional helicase domain. Further, C. elegans with mutations in the helicase domain of DCR-1 are viable and have wildtype miRNA levels.

While endo-siRNAs have been identified in several organisms, their precursors are ill-defined. D. melanogaster endo-siRNAs arise from loci predicted to form long, intra-molecular hairpins, as well as sense and antisense transcripts of overlapping genes. In plants, endo-siRNAs are produced from dsRNA either synthesized by an RNA-dependent RNA polymerase (RdRP) from a non-coding template RNA (tasiRNAs) or from natural antisense transcripts (nat-siRNAs). While a requirement of Dicer's helicase domain for processing these precursors has not been tested, current studies predict this highly conserved domain will be required for processive cleavage of dsRNA with blunt or 5′ overhangs in all organisms. Given the propensity of dsRNA to trigger the interferon response in mammalian cells, this function may be under tight regulation and require additional proteins.

Long dsRNA precursors of endo-siRNAs may not accumulate when Dicer cleavage is coupled to dsRNA synthesis by an RdRP, as in T. thermophile and S. pombe. For example, accumulation of dsRNA in S. pombe is only observed when Dicer is mutated to disrupt its RNase III activity, leading to a model whereby Dicer cleaves dsRNA as soon as it is synthesized by the RdRP. It seems likely that at least some C. elegans endo-siRNAs are produced by an RdRP-coupled mechanism, since DCR-1 is found in a complex that includes the RdRP RRF-3, and a 3′ to 5′ exonuclease, ERI-1. RRF-3 might synthesize dsRNA with heterogenous termini that are subsequently “polished” by ERI-1 to create blunt termini that require DCR-1's helicase domain for processing. Cleavage from the blunt terminus would yield a 26 nt endo-siRNA, while subsequent cleavages would produce shorter siRNAs. Interestingly, RRF-3, ERI-1, and DCR-1's helicase domain are all necessary for the production of 26G endo-siRNAs.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of producing siRNA in vitro comprising:
introducing at least one dsRNA molecule and at least one Dicer enzyme comprising a helicase domain to a sample to form a reaction mixture, wherein the dsRNA comprises blunt ends or at least one 5′ overhang; and
incubating the reaction mixture for a time sufficient to produce siRNA.

2. The method of claim 1, wherein the Dicer enzyme is a Drosophila Dicer-2 enzyme, a C. elegans Dicer enzyme or a human Dicer enzyme.

3. The method of claim 1, wherein the siRNA produced comprises a 3′ overhang.

4. The method of claim 3, wherein the siRNA produced comprises a 3′ overhang of 3 or 4 nucleotides.

5. The method of claim 1, wherein the dsRNA molecule of the reaction mixture does not comprise a 3′ overhang.

6. The method of claim 1, further comprising introducing ATP to the reaction mixture.

7. A method of modulating the production of siRNA in a subject comprising:
administering a mutated Dicer enzyme to the subject, wherein the Dicer enzyme comprises a mutation in the helicase domain of the Dicer enzyme, thereby modulating the production of siRNA in the subject.

8. The method of claim 7, further comprising administering at least one dsRNA molecule with a 3′ overhang to the subject.

9.-14. (canceled)

15. A method of screening for a candidate modulator that modulates siRNA production comprising the steps of:
(a) determining the production of siRNA in a sample comprising at least one dsRNA molecule and at least one Dicer enzyme comprising a helicase domain;
(b) exposing the sample comprising at least one dsRNA molecule and at least one Dicer enzyme comprising a helicase domain to the candidate modulator;
(c) determining the effect of the candidate modulator on the production of siRNA in the sample;
wherein a change in the production of siRNA in the sample after exposure to the candidate modulator is indicative of a modulator of siRNA production.

16. The method of claim 15, wherein the screening comprises a high-throughput screen.

17. (canceled)
